(19) United States
(12) Patent Application Publication
(10) Pub. No.: US 2020/0261439 A1
(43) Pub. Date: Aug. 20, 2020

(54) NICOTINE FORMULATION FOR ACTIVE MESH NEBULIZER

(71) Applicant: BN Intellectual Properties, Inc., Clearwater, FL (US)

(72) Inventor: Donald M. PELL, St. Petersburg, FL (US)

(21) Appl. No.: 16/547,478

(22) Filed: Aug. 21, 2019

Related U.S. Application Data
(60) Provisional application No. 62/806,217, filed on Feb. 15, 2019.

(51) Int. Cl. A61K 31/465; A61K 9/00

(52) U.S. Cl. CPC A61K 31/465; A61K 9/0078

(57) ABSTRACT

An aqueous nicotine solution for use in an active mesh nebulizer as part of a smoking cessation program, or as a substitute for tobacco use, includes nicotine in water with a nicotine concentration ranging from about 1.0 milligram (mg) nicotine per milliliter (ml) of water (e.g., 0.01% nicotine), to about 0.005 mg nicotine per milliliter of water (about 0.0005% nicotine). The pH of the aqueous nicotine solution ranges from about 3.5 to about 12 because the plume of particles of aqueous nicotine solution produced by the active mesh nebulizer are entrained with the inhaled air and do not impact the walls of the throat or lungs. The aqueous nicotine solution is free from metals because it is delivered to a user at room temperature, without heating or boiling.

### CROSS-REFERENCE TO RELATED APPLICATIONS

The present application is related to the following previously-filed patent applications, and incorporated the contents thereof in their entirety: U.S. Patent Application 62/806,217, filed on Feb. 15, 2019.

### BACKGROUND

Smoking cessation aids include transdermal patches and chewable products for delivering nicotine to a person seeking to eliminate a smoking habit. Transdermal patches release nicotine in a slow format, building up to a constant low level of nicotine in the body to reduce cravings for tobacco products such as cigarettes, cigars, or chewing tobacco. Chewable products release nicotine in an uneven release profile, with a first concentration of nicotine imparted to the body as the mouth and digestive system release nicotine from the chewable product into the digestive system and bloodstream, followed by a second concentration of nicotine with sporadic chewing of the chewable product. While transdermal patches provide a slow release, and chewable products have a more pronounced “high/low” concentration pattern, neither product mimics the nicotine uptake pattern from smoking tobacco products: neither mimics the pattern and concentration of nicotine delivery to which smokers are accustomed.

Electronic cigarettes provide nicotine to a user upon inhalation of a nicotine-containing formulation after the formulation is heated or boiled. Nicotine in solutions for electronic cigarettes has a high concentration (about 10 milligrams (mg) per milliliter (ml) of solution). The use of large amounts of nicotine in an electronic cigarette nicotine-containing solution makes tracking the dosage of nicotine received by an electronic cigarette user difficult in smoking cessation. Further, ingredients in a source solution used to mimic smoke exhalation by an electronic cigarette user are harmful to the central nervous system and cardiovascular system.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. The present disclosure may repeat reference numerals and/or letters in the various examples. This repetition is for the purpose of simplicity and clarity and does not in itself dictate a relationship between the various embodiments and/or configurations discussed.

The present disclosure relates to a liquid formulation or aqueous solution for delivering nicotine to a nicotine user without the inherent risks of other tobacco products. In some embodiments, the liquid formulation comprises water as a solvent, and nicotine in a concentration ranging from about 1 milligram per milliliter (1 mg/ml, or 0.1% according to e-cigarette terminology) to about 1/200 mg/ml (0.005 mg/ml or 0.0005% according to e-cigarette terminology). In some embodiments, the liquid formulation further comprises a water soluble flavoring component added to the water to modify the odor and/or flavor of the formulation after delivery of the compound to a user from an active mesh nebulizer. The active mesh nebulizer produces a plume of small-diameter droplets or particles of the liquid in the nebulizer capsule. Small volumes of particles have, after being converted to small-diameter particles, a large surface area which promotes evaporation of the droplet or of components in the droplet, which enhances the flavor and odor of the components in the droplet liquid. Nicotine has a flavor perceived to be strong and bitter according to many nicotine users. In some embodiments, a flavoring is added to the mixture to mask or moderate the strong or bitter flavor of nicotine. In some embodiments, the flavoring component added to the liquid formulation is a tobacco flavoring extract. In some embodiments, the flavoring component added to the liquid formulation includes menthol. In some embodiments, other flavoring components are added to the liquid formulation, including fruit flavorings, sweeteners, and/or the like.

Nicotine is a central nervous system stimulant in wide use around the world. Nicotine users frequently consume nicotine directly from tobacco products, which contain high natural levels of nicotine. Nicotine provides a user with a stimulating physiological response associated with the nicotine consumption. Nicotine is associated with elevated heart rate, greater alertness, and increased energy levels, improved mental retention, and suppression of weight gain. Nicotine use is habit forming. Nicotine users undergo withdrawal symptoms and cravings for nicotine after an initial dose of nicotine wears off. Habitual nicotine users exhibit tolerance for elevated doses of nicotine in addition to the withdrawal symptoms and cravings for additional nicotine after a nicotine dose wears off.

Tobacco leaves are the most common source of nicotine for nicotine consumption. Tobacco leaves are smoked in cigars, cigarettes, and pipes, and the nicotine in the tobacco leaves is absorbed by inhaling the tobacco smoke. Nicotine is also directly absorbed from tobacco leaves by swallowing nicotine released from tobacco held in the mouth and softened with saliva (e.g., buccal absorption, or sub-lingual absorption). Tobacco consumption is associated with many negative health outcomes. Tobacco contains several toxic and carcinogenic compounds. Tobacco use is associated with, among other conditions, elevated risks of cardiovascular disease, pneumonia, emphysema and respiratory illness, cataracts and eye disease, and various cancers including mouth, larynx, esophagus, stomach, pancreas, cervix, and kidney cancers. Decreasing the amount of tobacco use for individual patients, and for large populations, is has been shown to effectively decrease the frequency of these and other illnesses associated with tobacco consumption.

Smoking and tobacco cessation therapies to decrease tobacco consumption include the use of tobacco-free nicotine-containing materials to replace tobacco products. Tobacco-free nicotine-containing materials in tobacco cessation therapies are sometimes ineffective at replacing tobacco products because the tobacco-free nicotine-containing products do not produce a same brain response or a same physiological response as the targeted tobacco products. Because users crave not only the elevated nicotine levels, but also the sharp nicotine release profile associated with tobacco products, tobacco-free nicotine-containing products which do not produce tobacco-release profiles similar to tobacco products are only partially effective at smoking and tobacco cessation.

Smoking and tobacco cessation products include transdermal nicotine patches and nicotine gum (nicotine chewables). Transdermal patches have a slow nicotine release rate and a well-characterized blood serum level of nicotine, but do not provide nicotine users with a sudden surge in nicotine concentration associated with, e.g., smoking or chewing tobacco. Nicotine gum provides a somewhat more rapid nicotine release rate because the nicotine is released with the softening of the gum associated with chewing, and larger quantities of nicotine are absorbed by swallowing the nicotine-rich saliva, or by direct absorption thorough the tissues of the mouth. Nicotine dosage with nicotine gum is more difficult to regulate than with nicotine patches because the amount of nicotine released from the gum and absorbed into the bloodstream depends on the amount of chewing and swallowing by a nicotine user. Neither transdermal patches nor nicotine gum reproduce the rapid blood serum concentration increase associated with smoking or chewing tobacco products.

Electronic cigarettes produce a nicotine-containing vapor or droplet cloud which is absorbed into the bloodstream through the lungs. The nicotine from an electronic cigarette is released by heating an electronic cigarette nicotine-containing liquid formulation (electronic cigarette source solution) to boiling, or near-boiling, and releasing the vapors into the mouth and lungs of the user as the user inhales. Inhaling heated gases or liquids into the lungs over long periods of time is believed to put the lung and throat tissues at elevated risk of damage. The heating element which heats the electronic cigarette source solution contains metals such as cadmium (Cd), copper (Cu), nickel (Ni), and iron (Fe). Upon heating, some metal atoms transfer into the electronic cigarette source solution or the electronic cigarette vapor stream and into the electronic cigarette user's mouth and lungs. Ingestion/inhalation of metal from electronic cigarette heating elements is believed to be associated with elevated risk of illness.

While electronic cigarettes provide a nicotine release rate that is similar to the nicotine release rate of smoking tobacco products, and without the accompanying toxic and/or carcinogenic compounds in tobacco, [1] elevated levels of nicotine are associated with abnormal prenatal development in pregnant women, and heart arrhythmia in some persons, and [2] nicotine dosing with electronic cigarettes is extremely difficult to regulate because the amount of nicotine absorbed by a user is influenced by factors including the duration of the inhalation, the amount of time the inhaled material is retained in the lungs, the particle size of droplets of material, and the amount of material which impacts tissues in the mouth and throat, rather than the lungs. Electronic cigarette source solutions contain high nicotine concentrations to mimic the nicotine uptake pattern associated with smoking tobacco. Electronic cigarettes are not suitable for tobacco or smoking cessation because of the dosing accuracy issue presented above.

The present disclosure relates to a nebulizer source solution, an aqueous nicotine solution, or a nicotine-containing liquid formulation, suitable for use in an active mesh nebulizer to produce a strong brain response from the inhaled nicotine. The nebulizer source solution has a concentration of nicotine ranging from about 1 milligram per milliliter (1 mg/ml) to about 1/200 mg/ml (0.005 mg/ml), although other concentrations of nicotine are also within the scope of the present disclosure. In the electronic cigarette industry, an electronic cigarette source solution nicotine concentration is described as having a percentage “%” of nicotine therein as follows: a 50 mg/ml solution of nicotine in water is described as being a “5% nicotine” solution, and a 40 mg/ml solution of nicotine in water is described as being a “4% solution” of nicotine. Nicotine concentrations in nebulizer source solutions range, using the aforementioned electronic cigarette industry terminology, from 0.1% nicotine (e.g., 1 mg/ml of nicotine in water) to 0.0005% (0.005 mg/ml of nicotine in water). Concentrations of nicotine in electronic cigarette source solutions are unsuitable for use in an active mesh nebulizer because the greater delivery efficiency of an active mesh nebulizer produces near-immediate severe bronchospasm in a user. Concentrations of nicotine in nebulizer source solutions are unsuitable for use in an electronic cigarette because the total delivered dose of nicotine is too low to suppress nicotine withdrawal, suppress nicotine craving, and produce an appreciable blood serum concentration rise in a user.

An active mesh nebulizer uses a vibrating piezoelectric mesh in direct contact with a liquid solution to produce a plume of particles (liquid droplets, or droplets) of the liquid solution without heating or boiling. Active mesh nebulizers are sometimes configured to produce plumes of particles with diameters ranging from about 1.5 to about 5 micrometers (microns, μm). Particles having a diameter greater than about 5 microns have sufficient mass that, upon inhalation, the particles strike the walls of the mouth, throat, and upper branches of the lungs, where absorption rates are low, as compared to the absorption rates of inhaled droplets in the lower branches of the lungs and the alveolar sacs. Particles generated with a diameter ranging from about 1.5 to 5 microns are small enough to be carried, with little or no contact against the walls of the mouth, throat, and lungs, directly into the alveolar sacs in the lungs, where the particles are absorbed directly into the bloodstream.

Upon theory and belief, particle size is not constant subsequent to inhalation from an active mesh nebulizer. The particles produced by an active mesh nebulizer are sufficiently small that, after inhalation into the throat and lungs, the particles absorb liquid and increase in size. The aforementioned size increase is believed to be sufficient to dilute any acid or base molecules or ions present in the droplets, making the droplets less likely to produce a cough reaction or throat/lung irritation. Many electronic cigarette source solutions undergo a pH modification to a pH ranging from 6.5 to 8 in order to help a user tolerate the electronic cigarette plume without mouth or throat irritation or coughing. Electronic cigarette source solutions also undergo pH modification to a pH ranging from 6.5 to 8 in order to promote absorption of the delivered nicotine. A nebulizer source solution does is tolerated in the mouth throat, and lungs, and is readily promoted into the bloodstream, over a wide pH range because of the smaller particle size generated by the active mesh nebulizer. A plume having sufficiently small particle sizes does not make contact with the mouth or throat tissues to trigger coughing. Thus, a nebulizer source solution has, in some embodiments, a pH ranging from about 3.5 to about 12 with no negative impact on the lungs or nicotine absorption when delivered form an active mesh nebulizer.

In some embodiments, optional pH modification is accomplished by adding a pH modifier such as an acid to the source solution to lower the pH. In some embodiments, the pH modification is accomplished by adding a pH modifier such as a base to the source solution to raise the pH. In some embodiments, the pH modification is accomplished by adding, in addition to an acid or a base, a buffering element to the source solution to hold the pH constant.

The pH of a nebulizer source solution does not need to be modified in a manner similar to the modification of the pH of an electronic cigarette source solution. Thus, a nebulizer source solution pH ranges from pH of about 3.5 to about 12 with no mouth irritation, throat irritation, or coughing by a user. Because the particles produced by an active mesh nebulizer are entrained with a flow of inhaled air to the alveoli, and do not strike the walls of the mouth, throat, or lungs, the active mesh nebulizer source solution does not irritate the lungs. The upper regions of the lungs (e.g., above the second branching point) are the locations where coughing is triggered by irritation of the lung tissue. Small particles such as are produced by an active mesh nebulizer do not contact the lung tissue above the second branching point in the lungs, bypassing the locations of the lungs which trigger coughing and expulsion of inhaled materials from the lungs. In some embodiments, the pH of the solution is adjusted between about 3.5 and about 12 according to the conditions suitable for, e.g., [1] preserving the nicotine in solution without spoilage, or [2] preserving a flavor added to the solution, or some other consideration.

High nicotine-concentration electronic-cigarette source solutions are not suitable for use in an active mesh nebulizer. Even if a high-nicotine concentration electronic cigarette source solution were to be dispensed to a user with an active mesh nebulizer, bypassing the upper portions of the lungs (e.g., above the second branching point, see above) to avoid triggering coughing, the high nicotine concentration is believed to trigger bronchospasm, or rapid constriction of the bronchi, making it difficult for a person to breathe because of the sudden large influx of nicotine into the deep lung tissue below the second branching point of the lungs. According to theory and belief, a nicotine concentration threshold of a nebulizer source solution which does not trigger bronchospasm (e.g., a bronchospasm threshold) in a user of an active mesh nebulizer is not greater than 1.0 mg nicotine per milliliter of water (e.g., threshold≤1 mg/ml nicotine in water, or <0.1% nicotine in water), and greater than 0.005 mg/ml (or, 0.0005% nicotine in water).

Upon theory and belief, a nebulizer source solution containing 1 mg/ml of nicotine (nicotine salt, or free base nicotine) delivered to the lungs using an active mesh nebulizer is capable of producing a stronger brain effect in a nicotine user than plumes of vapor or droplets produced by an electronic cigarette. Upon theory and belief, a nebulizer source solution containing 1 mg/ml of nicotine (nicotine salt, or free base nicotine) delivered to the lungs using an electronic cigarette does not produce a similarly strong brain effect. An electronic cigarette producing a plume of vapor and droplets using an electronic cigarette source solution having a concentration of nicotine ranging from 10 mg/ml (e.g., 1% nicotine) to about 50 mg/ml (e.g., 5% nicotine) is not believed to be able to produce a brain response comparable to a plume delivered from an active mesh nebulizer because of different delivery (absorption) efficiencies between the devices.

The greater efficiency of absorption associated with a plume of nicotine-containing droplets from an active mesh nebulizer indicates that a lower total nicotine concentration (whether free base nicotine or a dissolved nicotine salt) in the nebulizer source solution is consistent with safe delivery of nicotine to a user with reduced risk of accidental nicotine overdose or nicotine poisoning.

According to theory and belief, a nicotine solution having 1 mg nicotine/ml of solution (e.g., 0.1%, whether free base or dissolve nicotine salt) delivered to a nicotine user with a single inhalation plume from an active mesh nebulizer with a nebulization rate of 0.2 ml/minute for a period of 10 seconds produces a strong brain effect, including profound relaxation, with no desire for further nicotine inhalation for at between 2-3 hours. According to theory and belief, a nicotine solution having 1/40 mg nicotine/ml solution (e.g., 0.025% nicotine) is also able to produce nicotine satiety for a period of at least two hours (nebulization rate of 0.2 ml/min nebulization rate, nebulization time of 10 seconds) also produced a strong brain effect, with no desire for further nicotine inhalation for at least two hours.

According to theory and belief, the small particle size and increased delivery efficiency of droplets of the active mesh nebulizer nicotine source solution produces a brain response in less than 10 seconds, comparable to or faster than the response from smoking tobacco or using an electronic cigarette. In some embodiments, the brain response from nicotine absorption from an active mesh nebulizer nicotine source solution occurs within not more than 7 seconds.

A nicotine source solution containing nicotine (whether free base or nicotine salt) of approximately 0.01 mg/ml to about 1 mg/ml is able to be used, with a time-regulated active mesh nebulizer, as part of a smoking cessation program. In some embodiments, the lower total nicotine delivered to the body from a single inhalation form an active mesh nebulizer with time-regulated delivery is approximately 10 to 1000 times smaller than the total amount of nicotine delivered to the body from a single inhalation of an electronic cigarette. Nicotine-related illness are believed to be less likely to develop in nicotine users from active mesh nebulizer inhalation of nicotine solutions because the total blood serum level of nicotine is significantly less than the amount absorbed from smoking tobacco or from electronic cigarette source solutions. Further, upon theory and belief, active mesh nebulizer-delivered nicotine source solutions do not produce metal contamination delivered to the lungs or bloodstream.

Nicotine solutions delivered from an active mesh nebulizer are delivered at room (e.g., ambient) temperature, with no heating or boiling. Because of the high absorption efficiency, there is no second-hand “smoke” or exhaled material from the lungs after inhalation of a plume of nicotine from an active mesh nebulizer. Thus, flavorings or nicotine or other compounds in the active mesh nebulizer source solution do not impact health or well-being of persons around the active mesh nebulizer.

Nicotine-containing source solutions consistent with the above description are suitable for tobacco or smoking cessation programs by providing high (e.g., around 1 mg/ml) nicotine concentrations to persons with strong nicotine tolerance and strong smoking habits, and solutions with lower concentrations of nicotine to persons undergoing a weaning or withdrawal process, while still providing the rapid brain response consistent with smoking to prevent a user from switching back to tobacco products and leaving the smoking cessation program.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A composition of matter, comprising:
water; and
nicotine, wherein a nicotine content in the water ranges from 0.005 milligram (mg) of nicotine per milliliter (ml) of water, to 1 mg nicotine per ml of water.

2. The composition of matter of claim 1, wherein a pH of the composition of matter ranges from 3.5 to 12.

3. The composition of matter of claim 2, wherein the pH of the composition of matter ranges from 3.5 to 7.

4. The composition of matter of claim 2, wherein the pH of the composition of matter ranges from 7 to 12.

5. The composition of matter of claim 1, wherein the nicotine content in water ranges from 0.005 mg/ml to 0.01 mg/ml.

6. The composition of matter of claim 1, wherein the nicotine content in water ranges from 0.01 mg/ml to 0.05 mg/ml.

7. The composition of matter of claim 1, wherein the nicotine content in water ranges from 0.05 mg/ml to 0.1 mg/ml.

8. The composition of matter of claim 1, wherein the nicotine content in water ranges from 0.1 mg/ml to 0.5 mg/ml.

9. The composition of matter of claim 1, wherein the nicotine content in water ranges from 0.5 mg/ml to 1 mg/ml.

10. A composition of matter for use in an active mesh nebulizer, comprising:
an aqueous nicotine solution having a nicotine concentration not greater than a bronchospasm threshold, wherein the bronchospasm threshold is not greater than 1 milligram nicotine per milliliter of water.

11. The composition of matter of claim 10, wherein further comprising a water-soluble flavoring.

12. The composition of matter of claim 11, wherein a pH is between 3.5 and 12.

13. The composition of matter of claim 12, wherein the pH is between 5 and 9.

14. The composition of matter of claim 12, wherein the pH is between 6 and 8.

15. The composition of matter of claim 14, further comprising a pH modifier configured to reduce the pH to between 6 and 8.

16. A composition of matter, comprising:
an aqueous nicotine solution having a pH between 3.5 and 12 and a nicotine concentration not greater than 1 milligram per milliliter (1 mg/ml, or 0.1% nicotine) and not less than 1/200 mg/ml (0.005 mg/ml, or 0.0005% nicotine).

17. The composition of matter of claim 16, wherein the aqueous nicotine solution has a pH between 3.5 and 7 and a nicotine concentration not less than 1/200 mg/ml (0.005 mg/ml, or 0.0005% nicotine) and not more than 0.1 mg/ml (or, 0.01%).

18. The composition of matter of claim 16, wherein the aqueous nicotine solution has a pH between 7 and 12 and a nicotine concentration not less than 0.1 mg/ml (or, 0.01%) and not less than 1 mg/ml (or, 0.1%).

19. The composition of matter of claim 16, further comprising a flavoring agent configured to mask a flavor of nicotine in the composition of matter.

20. The composition of matter of claim 16, wherein the aqueous nicotine solution has a pH between 6 and 8 and a nicotine concentration not less than 1/200 (0.005 mg/ml, or 0.0005%) mg/ml nicotine and not more than 0.5 mg/ml (or, 0.05%) nicotine per milliliter of water.
